(19) United States
(12) Patent Application Publication
(10) Pub. No.: US 2004/0185434 A1
(43) Pub. Date: Sep. 23, 2004

(54) Detecting microbial contamination in animal by-products

(76) Inventors: W. Wade Robey, Excelsior, MN (US); Alison M. Jones, Eden Prairie, MN (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/394,951

(22) Filed: Mar. 21, 2003

(51) Int. Cl. C12Q 1/70; C12Q 1/68; G01N 33/53; G01N 33/569; G01N 33/554

(52) U.S. Cl. 435/5; 435/6; 435/7.22; 435/7.32; 435/7.31

(57) ABSTRACT

The invention provides for methods of determining the presence, absence, or amount of microbial contamination in an animal by-product. The invention further provides for methods of monitoring animal by-products before, during, or after processing of the animal by-product into, for example, feed. The invention also provides for articles of manufacture for carrying out the claimed methods.

### TECHNICAL FIELD

This invention relates to animal by-products, and more particularly to methods of detecting microbial contamination in animal by-products.

### BACKGROUND

Meat, poultry, and fish by-products have commercial value and contribute significantly to the profits of slaughter operations. The use of by-products also reduces the overall environmental impact of processing operations. For example, pet food can be produced from viscera, gelatin can be produced from head pieces, meat meal can be produced from hoofs, chicken parts, bone and horn, glue can be produced from hides, and blood meal and small goods such as adhesives can be produced from blood. In addition, poultry feather and hog hair are rendered to convert keratin into amino acids.

The United States produces an average of seven million tons of rendered products annually with an estimated value of $3 billion. Rendering involves cooking, separating and drying processes that result in edible (suitable for human consumption) and inedible (not suitable for human consumption) animal by-products being made into useful commodities. Edible rendering facilities process fatty animal tissue into edible fats and proteins. The inedible rendering plants produce tallow and grease, which are used in livestock and poultry feed, soap, and production of fatty acids.

Processors of meat, fish, poultry, and of the subsequent animal by-products resulting therefrom, must operate in a manner that protects human health and the environment while maintaining the highest food safety standards. Many experts believe that the mad cow disease that developed in the United Kingdom was caused by using feed that contained animal by-products from scrapie-infected sheep. Other experts believe that mad cow disease was spread by causal agents in animal by-products from beef that were used for cattle feed. Consumption of beef containing residual brain or spinal cord tissue or the use of other beef products derived from “mad cows” in turn caused a human disorder affecting more than 100 individuals in Europe termed variant Creutzfeldt-Jakob. In 1994, the U.S. Food and Drug Administration issued a proposed rule declaring that specific offal from adult (more than 12 months old) sheep and goats is not generally recognized as safe for use in ruminant feed and is unapproved food additive when added to ruminant feed. The proposed rule defined “specific offal” as any tissue from the brain, spinal cord, spleen, thymus, tonsil, lymph nodes, or intestines of sheep or goat. Further, a 1998 ban in the UK on the practice of feeding processed ruminant protein to cattle has resulted in a steady reduction in the number of cases of mad cow disease detected in the cattle.

In view of the above, methods for detecting microbial contamination in animal by-products are useful so that illness or disease can be avoided in humans and animals that are consuming the animal by-product.

### SUMMARY

The invention provides for methods for determining the presence, absence, or amount of microbial contamination in an animal by-product. The invention further provides for methods of monitoring animal by-products before, during, or after processing of the animal by-product.

In one aspect, the invention provides for a method of determining the presence, absence, or amount of microbial contamination in an animal by-product. The method includes the steps of providing a sample from the animal by-product, and determining the presence, absence, or amount of microbial contamination in the sample. Typically, the presence of microbial contamination in the sample indicates the presence of microbial contamination in the animal by-product. The microbial contamination can be by one or a few species or genera of microbes, or can be by many species or genera of microbes.

In one embodiment, the microbes are taxonomically and phylogenetically identified. Generally, microbial contamination can include microbes that are bacteria, fungi, viruses, or protozoa. Representative bacterial microbes include those from the Staphylococcus, Streptococcus, Pseudomonas, Escherichia, Bacillus, Brucella, Chlamydia, Clostridium, Shigella, Mycobacterium, Agrobacterium, Bartonella, Borellia, Bradyrhizobium, Ehrlichia, Haemophilus, Helicobacter, Heliobacter, Lactobacillus, Neisseria, Rhizobium, Streptomyces, Synechococcus, Zymomonas, Synechocyotis, Mycoplasma, Yersinia, Vibrio, Burkholderia, Franciscella, Legionella, Salmonella, Bifidobacterium, Enterococcus, Enterobacter, Citrobacter, Bacteroides, Prevotella, Xanthomonas, Xylella, and Campylobacter genera; representative fungal microbes include those from the Aspergillus, Colletrotrichum, Cochliobolus, Helminthosporium, Microcyclus, Puccinia, Pyricularia, Deuterophoma, Monilia, Candida, and Saccharomyces genera; representative viral microbes include those from the Coronaviridae genus; and representative protozoan microbes include those from the Acanthamoeba, Cryptosporidium, and Tetrahymena genera.

Generally, animal by-products include hair, feathers, bone, viscera, blood, heads, feet, beaks, offal, and meat scraps. A sample used in the methods of the invention can be obtained from any of the above-listed animal by-products. An animal by-product can be a processed animal by-product. Representative processed animal by-products are fishmeal, feathermeal, blood meal, poultry litter, and bone meal. A sample used in the methods of the invention can be obtained from any of the above-listed processed animal by-products. A single sample can be obtained from any of the animal by-product or processed animal by-product identified herein, or a plurality of samples can be obtained. In one embodiment, the plurality of samples can be pooled.

Microbial contamination can be detected using a number of methods. For example, the determining step can include microbial culturing and colony identification, histological analysis, immunological analysis, genetic fingerprinting, ribosomal genotyping, and/or cpn60 genotyping. In general, the method used to determine the presence, absence, or amount of microbial contamination can be specific for one or a few species or genera of microbes or can be universal for many species or genera of microbes.

Methods of the invention can also include providing a control sample, and determining the amount of microbes in the control sample. Such a control sample can be a known amount of microbes or of a particular species of microbe.

In another aspect, the invention provides a method for monitoring animal by-products before, during, or after processing the animal by-product. Such a method includes providing a sample from the animal by-product, determining the presence, absence, or amount of microbial contamination in the sample, and tailoring the processing based upon the presence, absence, or amount of the microbial contamination.

For example, when the sample is obtained before the processing of the animal by-product, the tailoring step includes abandoning the processing, or expediting the processing. For example, when the sample is obtained during the processing of the animal by-product, the tailoring step includes abandoning the processing, expediting the processing, or repeating all or a portion of the processing. For example, when the sample is obtained after the processing of the animal by-product, the tailoring step can include repeating all or a portion of the processing. The tailoring step can be based on the presence or absence of microbial contamination, the amount of the microbial contamination, or both.

In yet another aspect of the invention, there is provided an article of manufacture that includes at least one microbial antibody that is attached to a solid support, and instructions for collecting a sample from an animal by-product and for determining the presence, absence, or amount of microbial contamination in the sample. The article of manufacture can further include an indicator molecule. In one specific embodiment, the solid support is a dipstick.

In still another aspect of the invention, there is provided an article of manufacture that includes at least one oligonucleotide that is complementary to nucleic acid sequences from one or a few number of microbial species; and instructions for collecting a sample from an animal by-product and for determining the presence, absence, or amount of microbial contamination in the sample. Alternatively, an article of manufacture of the invention can include at least one oligonucleotide that is complementary to nucleic acid sequences from microbial species of at least two genera and the above-described instructions.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### DESCRIPTION OF DRAWINGS

FIG. 1 is a diagram illustrating the flow of edible animal by-product processing.

FIG. 2 is a diagram illustrating the flow of inedible animal by-product processing.

Like reference symbols in the various drawings indicate like elements.

### DETAILED DESCRIPTION

The invention provides for methods for determining the presence, absence, or amount of microbial contamination in an animal by-product. As used herein, animal by-products are generated during animal processing and rendering. Animal by-products include hair, feathers, bone, viscera, blood, heads, feet, beaks, offal, and meat scraps. Animal by-products also can be processed into a variety of products, many of which are used as components of animal feed. Such processed animal by-products include, but are not limited to, fishmeal, feathermeal, bone meal, blood meal, and poultry meal. The invention further provides for methods of monitoring animal by-products before, during, or after processing of the animal by-product.

Animal Processing

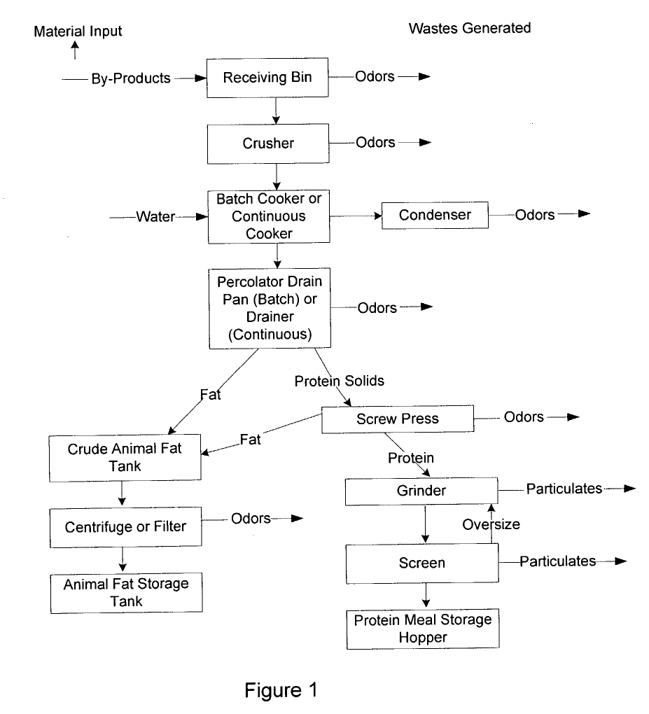

The diagram in FIG. 1 illustrates the flow of edible animal by-product processing. Edible animal by-products such as brain, heart, kidney, liver, pancreas, spleen, thymus, tongue, and tripe are chopped or ground into small pieces, and then cooked. As the material is heated, moisture and fats are released. The proteinaceous solids are separated from the melted fat and water by centrifugation. The edible fat is then separated from the water with additional centrifugation. The water is discharged as sludge, and the fat is pumped to storage.

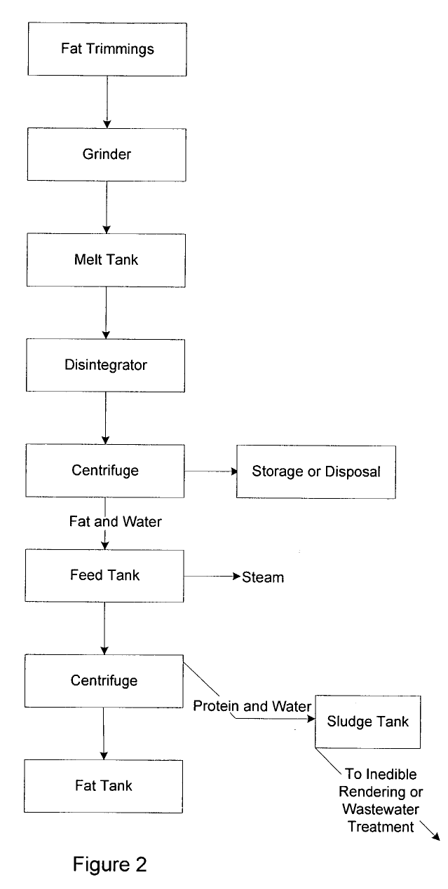

The diagram in FIG. 2 illustrates the flow of inedible animal by-product processing. Inedible animal by-products include hair, feathers, feet, and bone. Inedible rendering is performed by a wet or dry processing. Wet methods separate the fat from the raw materials by boiling in water. Water and live steam are used to cook the raw substances for fat separation. Dry rendering dehydrates the matter to release the fat. Following dehydration, the melted fat and protein solids are separated. At present, only dry rendering is used in the U.S. Wet rendering is no longer used due to its high-energy consumption and related costs, as well as adverse effects on fat quality.

Biogenic amines are volatile amines that are produced as a result of the breakdown of amino acids, typically upon death of an animal. Generally, biogenic amines are produced as a result of decarboxylation of amino acids during protein fermentation. Feed-borne biogenic amines are most commonly synthesized by spoilage microorganisms and are usually considered to be potential toxins. Therefore, not only are the presence of pathogenic microorganisms in animal feed of concern, any microorganism that is able to decarboxylate amino acids also is of concern. The most significant biogenic amine is histamine, which is produced by the breakdown of histidine. Other significant biogenic amines are putrescine, which is produced by the breakdown of glutamine, and cadaverine, which is produced by the breakdown of lysine. The levels of biogenic amines in fish and crustaceans can be used to indicate the degree of decomposition, so that the higher the concentration, the greater the amount of bacteria decomposition that has occurred.

The following sections describe the processing of animal by-products into various components of animal feed.

Fishmeal

High quality fishmeal is recognized by animal nutritionists as an excellent source of protein, energy, minerals and vitamins. In addition, the proteins in fishmeal contain all the essential amino-acids at significant concentrations. The majority of the fishmeal produced is included in commercial diets for poultry, swine, dairy cattle, mink and fish. It is of special value for young animals, for example in broiler starter diets, diets for early weaned pigs, and diets for farmed fish. In addition to proteins, fishmeal contains a “growth factor” necessary in animal production. Further, fishmeal supplementation may be a practical method to enhance fertility in beef cows, since recent studies in lactating dairy cows have shown that fishmeal supplementation increases conception rates by 10 to 20%.

Worldwide, millions of tons of fishmeal are produced annually. Fishmeal can be made from almost any type of fish but is generally manufactured from two main types. These two types of fish differ both in their ability to store oil as well as where in the body oil is stored.

The first type includes a group referred to as “lean fish.” This includes such species as cod and haddock. In these species the oil is stored primarily in the liver. The flesh (fillets) contain very little oil. Fishmeal from this type of fish has a low oil content (2 to 6%) since the livers are removed before processing. Of course, if the livers are added back, or the whole fish is used, the oil content would be higher. The whole fish is not usually used since cod and haddock are prized for the fillets. Since the fillets are used for human consumption, the fishmeal from these lean fish are made principally from the offal (white fish frames) remaining after filleting. White fishmeal constitutes only 10% of the world fishmeal production.

The second type of fish used to manufacture fishmeal stores oil in certain parts of the flesh. They are high oil fish and, unlike the lean fish, are not prized for their fillets. They are commonly referred to as “industrial fish.” Such species as herring, menhaden, anchovy, pilchard, sardines and mackerel fall into this category. Approximately 90% of the world fishmeal production is from these high oil species.

Fishmeal is made by cooking, pressing, drying and grinding the fish. When no oil needs to be removed, such as with lean fish, the pressing stage is often omitted. During cooking, the fish move through a long, steam-jacketed, screw conveyor cylinder. Cooking coagulates the proteins and is a critical process responsible for sterilizing the product and preparing it for liquor (a mixture of oil, water and protein) removal. Once cooked, the liquor is removed by pressing. The solid residue that remains after pressing is called “presscake.” The liquor is centrifuged to remove the oil. This oil is often further refined before being transported to storage tanks. Prior to storage, it is essential to add an antioxidant to the oil. The antioxidant will stabilize the oil so that oxygen will not cause damage during storage. The stored oil must not come into contact with air, heat or light in order that its quality be maintained until it can be incorporated into feed for poultry, pets, fish or other uses.

The liquid removed from presscake is referred to in the processing industry as “stickwater.” This liquid may contain as much as 20% soluble protein and is therefore valuable. The stickwater is evaporated to a thick syrup containing 30 to 50% solids. This material can be sold as “condensed fish solubles” or it can be added back to the presscake and dried with it. Therefore, presscake meal or whole meal (where all of the solubles have been added back) can be purchased.

The fishmeal is then dried so that the moisture content is low enough to allow the fishmeal to be stored and transported without mold or bacterial growth. If overdrying occurs, the fishmeal can be scorched and the nutritional value of the meal will be adversely affected. Drying can be either direct or indirect. Direct drying is the most rapid and requires very hot air to be passed over the meal as it is rapidly tumbled in a cylindrical drum. Indirect drying requires a stream-jacketed cylinder or a cylinder containing steam-heated discs that tumble the meal. Once the fishmeal is dried, it is ground, screened to the correct particle size, and packed in bags or stored in silos for bulk delivery to companies throughout the world.

The main industrial fish harvested in the U.S. is menhaden and is taken from the Gulf of Mexico and the Atlantic Ocean. In fact, 98% of the fish oil produced in the U.S. is from the menhaden, a high oil species. Menhaden fishmeal is commercially available (Sea-Lac, Omega Protein, Hammond La.). Smaller quantities of fishmeal produced in the U.S. are made from herring, redfish, and white fish. Fishmeal produced from these fish is low in oil, and comparatively higher in ash than the fishmeal from menhaden because of the relatively large amount of bone these fish contains compared to the amount of muscle.

Feather Meal

Feather meal consists of ground poultry feathers and is produced by hydrolyzing clean, undecomposed feathers from slaughtered poultry. Feathers collected from a slaughter facility are cooked at 140-150° for 30 minutes under live steam pressure. After cooking, the product is then dried and ground to produce a free flowing meal. Variability of feather meal between batches and between plants can be quite high due to differences in the animal by-products included (heads, feet, skin, etc), but the average feather meal product contains 85% crude protein, 10% fat and 4-7% moisture. The most important factor affecting the quality of feather meal is the extent of hydrolyzation. If less then 75% of the crude protein content is digestible by the pepsin digestibility method, then hydrolyzation was incomplete and protein quality is reduced. When fed alone or in high concentrations (greater than 40% of a feed), palatability problems have been observed. However, when fed as part of a protein supplement for complete diets (feedlot situation) or when incorporated in a molasses-based liquid supplement, palatability problems have not arisen. The protein in feather meal is digested more slowly in the rumen than other protein sources.

Feather meal also has a higher rumen bypass potential than traditional protein sources. Bypass protein refers to the amount of dietary protein escaping microbial degradation in the rumen and arriving at the small intestine. Efficiency of dietary protein use by the ruminant animal is dependent upon the amount of high quality protein appearing at the primary site of absorption, which is the small intestine. This higher escape potential allows producers to decrease, but not necessarily eliminate, the amount of expensive natural proteins fed. The incorporation of urea to boost crude protein content and provide a readily available source of nitrogen for the rumen microbes is used to compliment feather meal in cattle diets and supplements.

Bonemeal

Bonemeal is the most natural form of calcium and phosphorus available for animal health. This dried and ground product is sterilized by cooking with steam under pressure. This material is produced exclusively from bones.

Bloodmeal

Whole blood from animal slaughterhouses is used to produce as blood meal, which is a valuable ingredient in animal feed due to its high lysine content. The efficient recovery and segregation of blood is an important means of reducing the pollution loads in wastewaters, since blood is a highly polluting substance. An operation with an efficient recovery system will have a 40% lower polluting load than one that allows blood to flow to the wastewater stream.

Poultry Litter

Poultry litter, particularly broiler litter, is high in protein (25%) and minerals, with maintenance levels of energy (45% total dietary nitrogen (TDN)). Broiler litter typically has high concentrations of calcium and potassium. Most broiler houses are cleaned once each year, and bedding (e.g., wood shavings) makes up only a small part of the litter. Much of the crude protein is in the form of non-protein nitrogen that is better utilized when mixed with moderate-to high-energy feeds. Broiler litter should be analyzed for soil contamination prior to feeding and only litter having limited soil contamination should be fed to cattle. Feed-grade poultry litter should contain no more than 28% ash.

Broiler litter can be hauled in dump trucks and handled with front-end loaders. As soon after cleanout as possible, litter should be deep-stacked and allowed to go through a heat process. Proper stacking eliminates ammonia, which improves palatability and dry matter intake. Litter can be stored in buildings or outside on well-drained sites. In either storage location, the litter should be covered with plastic to limit oxygen access and help prevent overheating.

Broiler litter is often mixed with an energy source and fed to ruminants as a component of the feed ration. The energy added to the litter can be from corn, wheat, oats, whole cottonseed-seed (up to 25% of the ration), or from other by-products such as soybean hulls, cookie scraps, or bread.

Offal

Edible offal is the edible tissues and organs other than muscles and fat from slaughtered animals. Edible offal from slaughtered mammals includes brain, heart, kidney, liver, pancreas, spleen, thymus, tongue and tripe, all of which may be consumed.

Edible offal from poultry includes liver, gizzard, heart, skin, all of which may be consumed. Poultry offal meal is a combination of all poultry by-products processed together in the same proportions as they occur in the processing plant. The composition can be quite variable from plant to plant and batch to batch, depending upon what is being included. Poultry offal meal is suitable to be used in poultry diets.

The invention provides for methods of monitoring animal by-products before, during, or after processing of the animal by-product. Depending upon the presence, absence, or amount of microbial contamination, the processing step can be tailored to address the presence and amount of microbial contamination. For example, if a sample is obtained from an animal by-product that has not been processed and the presence of contaminating microbes is detected, the animal by-product can be processed expeditiously if, for example, high amounts of microbial contamination are detected, or not processed at all (e.g., discarded) if a high amount of microbial contamination is detected. If a sample is obtained during processing of the animal by-product into, for example, feed or a component thereof and microbial contamination is detected, the animal by-product can be processed expeditiously, not processed at all, or all or a portion of the processing can be repeated. If a sample is obtained from a processed animal by-product and microbial contamination is detected, the processing can be repeated in part or in full.

Samples and Sampling Methods

The methods described herein are capable of detecting the presence, absence, or amount of a microbe in a sample obtained from an animal by-product. Methods of the invention also can be used for monitoring animal by-products before, during, and after processing. The presence or absence of microbes can be determined in a sample from an animal by-product.

As used herein, “biological sample” refers to any sample obtained, directly or indirectly, from animal by-product, either before, during, or after being processed. Therefore, a sample useful in the methods of the invention includes any portion of the animal by-product, processed or otherwise, listed above. Sampling an animal by-product can include taking a single sample or taking multiple samples. Multiple samples can be pooled and analyzed together, or can be kept separate and analyzed individually. Since microbial contamination may not be uniform throughout animal by-products or processed animal by-products, it may be advantageous to obtain multiple samples using, for example, a systematic and/or stratified sampling arrangement (see, for example, Thompson, 1997, Ciba. Found. Symp., 210:161-72; and Park & Pohland, 1989, J. Assoc. Off. Anal. Chem., 72(3):399-404). The American Society for Testing and Materials (ASTM) publishes standards for various sampling methods. See, for example, ASTM Standards on Environmental Sampling, 2^{nd} Ed., 1997; ASTM Standards on Environmental Site Characterization, 2^{nd}, Ed., 2002; and ASTM Standard Practice for Aseptic Sampling of Biological Materials, 1999. In addition, resources are available that provide guidance in ensuring appropriate sample design and statistical analysis of results (see, for example, Anderson-Sprecher et al., 1994, J. Expo. Anal. Environ. Epidemiol., 4(2):115-31).

Without limitation, samples can be tissues such as brain or skin from an animal by-product, or fluid such as blood. Tissue samples can include biopsy samples or swabs of the tissue of interest. The tissue can be an animal by-product from any appropriate animal, such as a cow, pig, chicken, turkey, horse, goat, sheep, and fish, such as menhaden, herring, redfish, and white fish. The tissue of interest also can include an eye, a tongue, a hoof, a beak, a snout, a foot, a feather, an ear, a nose, the fur, and the skin.

Biological fluids can include bodily fluids (e.g., urine, lachrymal fluid, vitreous fluid, sputum, cerebrospinal fluid, sweat, lymph, saliva, semen, blood, or serum or plasma derived from blood) fluid such as a cell culture or a supernatant from a cell culture; and a fluid such as a buffer that has been used to obtain or resuspend a sample, e.g., to wash or to wet a swab in a swab sampling procedure. Biological samples can be obtained from an animal using methods and techniques known in the art. See, for example, Diagnostic Molecular Microbiology: Principles and Applications (Persing et al. (eds), 1993, American Society for Microbiology, Washington D.C).

Methods for sampling a tissue with a swab are known to those of skill in the art. Generally, a swab is hydrated (e.g., with an appropriate buffer, such as Cary-Blair medium, Stuart's medium, Amie's medium, PBS, buffered glycerol saline, or water) and used to sample an appropriate tissue for a microbe. Any microbe present is then recovered from the swab, such as by centrifugation of the hydrating fluid away from the swab, removal of supernatant, and resuspension of the centrifugate in an appropriate buffer, or by washing of the swab with additional diluent or buffer. The so-recovered sample may then be analyzed according to the methods described herein for the presence of a microbe. Alternatively, the swab may be used to culture a liquid or plate (e.g., agar) medium in order to promote the growth of any microbes for later testing. Suitable swabs include both cotton and sponge swabs; see, for example, those provided by Tecra®, such as the Tecra ENVIROSWAB®.

Methods for collecting and storing samples are generally known to those of skill in the art. For example, the Association of Analytical Communities International (AOAC International) publishes and validates sampling techniques for testing foods and agricultural products for microbial contamination. See also WO 98/32020 and U.S. Pat. No. 5,624,810, which set forth methods and devices for collecting and concentrating microbes. WO 98/32020 also provides methods for removing somatic cells, or animal body cells present at varying levels in certain samples.

In particular embodiments of the methods described herein, a separation and/or concentration step may be necessary to separate any microbes present from other components of a sample or to concentrate the microbe to an amount sufficient for rapid detection. For example, a sample suspected of containing a biological microbe may require a selective enrichment of the microbe (e.g., by culturing in appropriate media, e.g., for 4-96 hours, or longer) prior to employing the detection methods described herein. Alternatively, appropriate filters and/or immunomagnetic separations can concentrate a microbe without the need for an extended growth stage. For example, antibodies with binding affinity to a microbial polypeptide can be attached to magnetic beads and/or particles. Multiplexed separations, in which two or more concentration processes are employed, are also contemplated, e.g., centrifugation, membrane filtration, electrophoresis, ion exchange, affinity chromatography, and immunomagnetic separations.

The samples from the animal by-product can be used “as is,” or may need to be treated prior to application of the detection methods employed herein. For example, samples can be processed (e.g., by nucleic acid or protein extraction methods and/or kits known in the art) to release nucleic acid or proteins. In other cases, a biological sample can be contacted directly with PCR reaction components and appropriate oligonucleotide primers and probes.

Detection of Microbial Contamination

As used herein, “microbial contamination” refers to the presence of one or more bacteria, protozoa, viruses, and/or fungi. Microbial contamination is not limited to the presence of pathogenic microbes. Microbes that can contaminate animal by-products include the following examples of prokaryotic genera: Staphylococcus, Streptococcus, Pseudomonas, Escherichia, Bacillus, Brucella, Chlamydia, Clostridium, Shigella, Mycobacterium, Agrobacterium, Bartonella, Borellia, Bradyrhizobium, Ehrlichia, Haemophilus, Helicobacter, Heliobacter, Lactobacillus, Neisseria, Rhizobium, Streptomyces, Synechococcus, Zymomonas, Synechocyotis, Mycoplasma, Yersinia, Vibrio, Burkholderia, Franciscella, Legionella, Salmonella, Bifidobacterium, Enterococcus, Enterobacter, Citrobacter, Bacteroides, Prevotella, Xanthomonas, Xylella, and Campylobacter; the following examples of protozoa genera: Acanthamoeba, Cryptosporidium, and Tetrahymena; the following examples of fungal genera: Aspergillus, Colletrotrichum, Cochliobolus, Helminthosporium, Microcyclus, Puccinia, Pyricularia, Deuterophoma, Monilia, Candida, and Saccharomyces; and the following viral microbes: Coronaviridae.

The microbes in a sample can be evaluated and monitored using a number of methods. For example, the microbes in a sample can be cultured and colonies identified and/or enumerated. It has been estimated, however, that culturing typically recovers only about 0.1% of the microbial species in a sample (based on comparisons between direct microscopic counts and recovered colony-forming units). An improvement on culture-based methods is a community-level physiological profile. Such determinations can be accomplished by monitoring the capacity of a microbial community to utilize a suite of carbon sources with subsequent detection of the end product of this carbon metabolism by, for example, reduction of a tetrazolium dye. Profiling the physiology of a microbial community can yield qualitative (e.g., different patterns of reduced substrates) and semi-quantitative (e.g., spectrophotometric measurement of reduction) results. Biolog, Inc. (Hayward, Calif.) has commercialized a microtiter plate assay useful for determining the physiological profile of a complex microbial community. The BIOLOG method requires a standard inoculum density of metabolically active microorganisms, and assumes that all members of the community grow at the same rate so the utilization profile is not skewed by the metabolic capabilities of the fastest growers, and further assumes that the 95 substrates reflect the comprehensive substrate availability in the environment of interest.

Culture-independent methods to evaluate the microbes in a sample consist of extracting and analyzing microbial macromolecules from a sample. In general, useful target molecules are ones that, as a class, are found in all microorganisms, but are diverse in their structures, thereby reflecting the diversity of the microbes. Examples of target molecules include phospholipid fatty acids (PLFA), polypeptides, and nucleic acids. PLFA analysis is based on the universal presence of modified fatty acids in microbial membranes, and is useful as a taxonomic tool. PLFAs are easily extracted from samples, and separation of the various signature structures reveals the presence and abundance of classes of microbes. This method requires appropriate signature molecules, which often are not known or may not be available for the microbes of interest. In addition, the method requires that an organism's PLFA content does not change under different metabolic conditions. Another limitation to using PLFAs as target molecules is that widely divergent organisms may have the same signature set of PLFAs.

Other less direct measures can be made that can provide insight into the microbes in a sample. For example, tissues can be excised and histologically evaluated for the number, size, shape, mucosal-cell turnover and condition of the villi. The microscopic appearance of the villi can correlate with changes in the microbial ecology of the animal, as many of the resident organisms attach directly to the mucosa.

Techniques such as immunohistochemical analysis also can be employed as indicative measures of the presence of microbes in a tissue sample. An increased presence of leukocytic cytokines (lymphokines and monokines), in, for example, head tissue or blood by-products as well as the presence of immunoglobulins (e.g., IgM, IgG, or IgA) in such by-products can be examined and used to evaluate the microbes in a sample.

Various nucleic acid-based assays can be employed to examine the microbes in a sample. For example, some nucleic acid-based population methods use denaturation and reannealing kinetics to derive an indirect estimate of the percent (%) guanine and cytosine nucleotides (G+C) content of the DNA in the sample. This method has been used to characterize a bacterial community in the ileum and cecum of the GIT in poultry, and to examine how diet and other variables modulate the microbial communities in the GITs of animals (Apajalahti et al., 2001, Appl. Environ. Microbiol., 67:5656-67). The % G+C technique provides an overall view of the microbial community and is sensitive only to massive changes in the make-up of the community.

Genetic fingerprinting of a sample is another method that can be used to examine a sample for the presence of microbes. Genetic fingerprinting utilizes random-sequence oligonucleotide primers that hybridize with sequence-specificity to random sequences throughout the genome. Amplification results in a multitude of products. The distribution of amplification products is referred to as a genetic fingerprint. Particular patterns can be associated with microbes in the sample. Genetic fingerprinting, however, lacks the ability to conclusively identify specific microbial species.

Denaturing or temperature gradient gel electrophoresis (DGGE or TGGE) is another technique that can be used to examine a sample for the presence of microbes. As amplification products are electrophoresed in gradients with increasing denaturant or temperature, the double-stranded molecule melts and its mobility is reduced. The melting behavior is determined by the nucleotide sequence, and unique sequences will resolve into individual bands. Thus, a D/TGGE gel yields a genetic fingerprint characteristic of the microbial community, and the relative intensity of each band reflects the abundance of the corresponding microorganism. An alternative format includes single-stranded conformation polymorphism (SSCP). SSCP relies on the same physical basis as % G+C renaturation methods, but reflects a significant improvement over such methods.

In addition, the microbes in a sample can be evaluated using terminal restriction fragment length polymorphism (TRFLP). Amplification products can be analyzed for the presence of known sequence motifs using restriction endonucleases that recognize and cleave double-stranded nucleic acids at these motifs. For example, the enzyme Hhal cuts at 5′-GCGC-3′ sites. Using a fluorescently-labeled primer to tag one end of the amplification product and Hhal to digest the products, resolution of this mixture by electrophoresis will yield a series of fluorescent bands whose lengths are determined by how far a 5′-GCGC-3′ motif lies from the terminal tag. TRFLP profiles can be generated using a variety of restriction enzymes, and can be correlated with changes in the microbial population. For example, a TRFLP database for 16S rRNA sequences has been set up at Michigan State University to allow researchers to design experimental parameters (e.g., choice of enzyme and primer combinations). The principal advantages of TRFLP are its robustness and its low cost. Unlike D/TGGE, experimental conditions need not be stringently controlled since the profiles are size-based and thus can be generated by a variety of gel systems, including automated DNA sequencing machines. Alternative approaches include “amplified ribosomal DNA restriction analysis (AADRA)” in which the entire amplification product, rather than just the terminal fragment, is considered. AADRA, however, becomes unmanageable with communities containing many species.

Genotyping of 16S ribosomal DNA (rDNA) is another way to examine the microbes in a sample. 16S rDNA sequences are universal, composed both of highly conserved regions, which allows for the design of common amplification primers, and open reading frame (ORF) regions with sequence variation, which allows for phylogenetic differentiation. 16S ribosomal sequences are relatively abundant in the RNA form. In addition to amplification using oligonucleotide primers, genotyping of 16S rDNA can be performed using other methods including restriction fragment length polymorphism (RFLP) with Southern blotting.

Other targets for genotyping include genes encoding components of RNA polymerase, translation elongation factors, gyrase, and chaperoning. Such protein-encoding sequences may evolve more rapidly than those encoding structural RNAs. Thus, the sequences of protein-encoding sequences in closely related species may have diverged more in closely related species and may provide more discriminatory information. The choice of which target sequence to use depends on whether the sequences provide both broad coverage and discriminatory power. Ideally, the target should be present in all members of a given microbial community, be amplified from each member with equal efficiency using common primers, yet have distinct sequences. Multiple targets may in fact prove necessary for particular applications.

Chaperonin 60 (cpn60) nucleic acid sequences are particularly useful targets for genotyping and can be used to examine the microbes in a sample such as a sample comprising tissue from an animal. Chaperonin proteins are molecular chaperones required for proper folding of polypeptides in vivo. cpn60 is found universally in prokaryotes and in the organelles of eukaryotes, and can be used as a species-specific target and/or probe for identification and classification of microorganisms. Sequence diversity within this protein-encoding gene appears greater between and within bacterial genera than for 16S rDNA sequences, thus making cpn60 a superior target sequence having more distinguishing power for microbial identification at the species level than 16S rDNA sequences.

PCR oligonucleotide primers that universally amplify a 552-558 base pair (bp) segment of cpn60 from numerous microorganisms have been generated (see, for example, U.S. Pat. Nos. 5,708,160 and 5,989,821), and the nucleotide sequence of this region of cpn60 has been evaluated as a tool for microbial analysis. The utility of the sequence diversity in cpn60 has been demonstrated, in part, by cross hybridization experiments using nylon membranes spotted with cpn60 amplification products from typed strains probed with labeled amplification product from unknown isolates. By manipulating stringency conditions, hybridization can be limited to targets having >75% identity (e.g., >80%, >85%, >90%, >95% identify) to the unknown isolate. This level of cross hybridization allows for clear differentiation of species within genera.

Nucleic acid hybridization is another method that can be used to examine the microbes in an animal by-product. Probing amplification products with species-specific hybridization probes is one of the most powerful analytical tools available for profiling. The physical matrix for hybridization can be nylon membranes (e.g., macroarrays) or microarrays (e.g., microchips), incorporation of the hybridization probes into the amplification reaction (e.g., TaqMan or Molecular Beacon technology), solution-based methods (e.g., ORIGEN technology), or any one of numerous approaches devised for clinical diagnostics. Probes can be designed to preferentially hybridize to amplification products from individual species or to discriminate species phylogenetically.

The microbes in a sample also can be examined by cloning and sequencing microbial nucleic acids present in the sample. Cloning of individual nucleic acids into Escherichia coli and sequencing each nucleic acid gives the highest density of information but requires the most effort. Although sequencing nucleic acids is automated, routine monitoring of changes in the microbial profile of an animal by cloning and sequencing nucleic acids from the microorganisms still requires considerable time and effort.

Representative Nucleic-Acid Based Assays

PCR Assays

Nucleic acid-based methods for identifying and/or quantitating the amount of a microbe in a sample can include amplification of a nucleic acid. Amplification methods such as PCR provide powerful means by which to increase the amount of a particular nucleic acid sequence. Nucleic acid hybridization also can be included in determining the presence or absence of a microbe in a sample. Probing amplification products with species-specific hybridization probes is one of the most powerful analytical tools available for profiling. The physical matrix for hybridization can be a nylon membrane (e.g., a macroarray) or a microarray (e.g., a microchip), incorporation of one or more hybridization probes into an amplification reaction (e.g., TaqMan® or Molecular Beacon technology), solution-based methods (e.g., ORIGEN technology), or any one of numerous approaches devised for clinical diagnostics. As discussed above, probes can be designed to preferentially hybridize to amplification products from individual species or to discriminate specific species.

U.S. Pat. Nos. 4,683,202, 4,683,195, 4,800,159, and 4,965,188 disclose conventional PCR techniques. PCR typically employs two oligonucleotide primers that bind to a selected nucleic acid template (e.g., DNA or RNA). Primers useful in the present invention include oligonucleotide primers capable of acting as a point of initiation of nucleic acid synthesis within or adjacent to sequences. A primer can be purified from a restriction digest by conventional methods, or can be produced synthetically. Primers typically are single-stranded for maximum efficiency in amplification, but a primer can be double-stranded. Double-stranded primers are first denatured (e.g., treated with heat) to separate the strands before use in amplification. Primers can be designed to amplify a nucleotide sequence from a particular microbial species, or can be designed to amplify a sequence from more than one species. Primers that can be used to amplify a nucleotide sequence from more than one species are referred to herein as “universal primers.”

PCR assays can employ template nucleic acids such as DNA or RNA, including messenger RNA (mRNA). The template nucleic acid need not be purified; it can be a minor fraction of a complex mixture, such as a microbial nucleic acid contained in animal cells. Template DNA or RNA can be extracted from a biological or non-biological sample using routine techniques such as those described in Diagnostic Molecular Microbiology: Principles and Applications (Persing et al. (eds.), 1993, American Society for Microbiology, Washington D.C.). Nucleic acids can be obtained from any of a number of sources, including plasmids, bacteria, yeast, organelles, and higher organisms such as plants and animals. Standard conditions for generating a PCR product are well known in the art (see, e.g., PCR Primer: A Laboratory Manual, Dieffenbach and Dveksler (eds.), Cold Spring Harbor Laboratory Press, 1995).

Once a PCR amplification product is generated, it can be detected by, for example, electrophoresis and/or hybridization. One type of hybridization commonly used is a Southern blot. Southern blot hybridization between nucleic acid molecules is discussed in detail in Sambrook et al. (1989, Molecular Cloning: A Laboratory Manual, 2^{nd} Ed., Cold Spring Harbor Laboratory Press, Cold Spring Harbor, N.Y.; Sections 7.37-7.57, 9.47-9.57, 11.7-11.8, and 11.45-11.57).

If microbial contamination is present in a sample, a hybridization complex is produced between the microbial nucleic acid and an oligonucleotide probe. For oligonucleotide probes less than about 100 nucleotides, Sambrook et al. discloses suitable Southern blot conditions in Sections 11.45-11.46. The Tm between a sequence that is less than 100 nucleotides in length and a second sequence can be calculated using the formula provided in Section 11.46. Sambrook et al. additionally discloses prehybridization and hybridization conditions for a Southern blot that uses oligonucleotide probes greater than about 100 nucleotides (see Sections 9.47-9.52). Hybridizations with an oligonucleotide greater than 100 nucleotides generally are performed 15-25° C. below the Tm. The Tm between a sequence greater than 100 nucleotides in length and a second sequence can be calculated using the formula provided in Sections 9.50-9.51 of Sambrook et al. Additionally, Sambrook et al. recommends the conditions indicated in Section 9.54 for washing a Southern blot that has been probed with an oligonucleotide greater than about 100 nucleotides.

The conditions under which membranes containing nucleic acids are prehybridized and hybridized, as well as the conditions under which membranes containing nucleic acids are washed to remove excess and non-specifically bound probe can play a significant role in the stringency of the hybridization. Such hybridizations can be performed, where appropriate, under moderate or high stringency conditions. Such conditions are described, for example, in Sambrook et al. section 11.45-11.46. For example, washing conditions can be made more stringent by decreasing the salt concentration in the wash solutions and/or by increasing the temperature at which the washes are performed. In addition, interpreting the amount of hybridization can be affected, for example, by the specific activity of the labeled oligonucleotide probe, by the number of probe-binding sites on the template nucleic acid to which the probe has hybridized, and by the amount of exposure of an autoradiograph or other detection medium.

It will be readily appreciated by those of ordinary skill in the art that although any number of hybridization and washing conditions can be used to examine hybridization of a probe nucleic acid molecule to immobilized target nucleic acids, it is more important to examine hybridization of a probe to target nucleic acids under identical hybridization, washing, and exposure conditions. Preferably, the target nucleic acids are on the same membrane.

A nucleic acid molecule is deemed to hybridize to nucleic acids from a microorganism (e.g., a contaminating microbe) but not to control nucleic acids if hybridization to nucleic acid from a microorganism is at least 5-fold (e.g., at least 6-fold, 7-fold, 8-fold, 9-fold, 10-fold, 20-fold, 50-fold, or 100-fold) greater than hybridization to control nucleic acid. The amount of hybridization can be quantitated directly on a membrane or from an autoradiograph using, for example, a PhosphorImager or a Densitometer (Molecular Dynamics, Sunnyvale, Calif.).

Another form of hybridization involves the use of FRET technology. FRET technology (see, for example, U.S. Pat. Nos. 4,996,143, 5,565,322, 5,849,489, and 6,162,603) is based on the concept that when a donor fluorescent moiety and a corresponding acceptor fluorescent moiety are positioned within a certain distance of each other, energy transfer taking place between the two fluorescent moieties can be visualized or otherwise detected and quantitated. One or two oligonucleotide probes containing fluorescent moieties, can hybridize to an amplification product at particular positions determined by the complementarity of the oligonucleotide probe(s) to the target nucleic acid sequence. Generally, and depending on the particular strategy for detection (e.g., Molecular Beacon technology) hybridization of the oligonucleotide probe(s) to the amplification product at the appropriate positions generates a FRET signal. Detection of FRET can occur in real-time, such that the increase in an amplification product after each cycle of a PCR assay is detected and, in some embodiments, quantified.

Fluorescent analysis and quantification can be carried out using, for example, a photon counting epifluorescent microscope system (containing the appropriate dichroic mirror and filters for monitoring fluorescent emission in a particular range of wavelengths), a photon counting photomultiplier system, or a fluorometer. Excitation to initiate energy transfer can be carried out with an argon ion laser, a high intensity mercury arc lamp, a fiber optic light source, or another high intensity light source appropriately filtered for excitation in the desired range.

Fluorescent moieties can be, for example, a donor moiety and a corresponding acceptor moiety. As used herein with respect to donor and corresponding acceptor fluorescent moieties, “corresponding” refers to an acceptor fluorescent moiety having an emission spectrum that overlaps the excitation spectrum of the donor fluorescent moiety. The wavelength maximum of the emission spectrum of an acceptor fluorescent moiety typically should be at least 100 nm greater than the wavelength maximum of the excitation spectrum of the donor fluorescent moiety, such that efficient non-radiative energy transfer can be produced therebetween.

Fluorescent donor and corresponding acceptor moieties are generally chosen for (a) high efficiency Förster energy transfer; (b) a large final Stokes shift (>100 nm); (c) shift of the emission as far as possible into the red portion of the visible spectrum (>600 nm); and (d) shift of the emission to a higher wavelength than the Raman water fluorescent emission produced by excitation at the donor excitation wavelength. For example, a donor fluorescent moiety can be chosen with an excitation maximum near a laser line (for example, Helium-Cadmium 442 nm or Argon 488 nm), a high extinction coefficient, a high quantum yield, and a good overlap of its fluorescent emission with the excitation spectrum of the corresponding acceptor fluorescent moiety. A corresponding acceptor fluorescent moiety can be chosen that has a high extinction coefficient, a high quantum yield, a good overlap of its excitation with the emission of the donor fluorescent moiety, and emission in the red part of the visible spectrum (>600 nm).

Representative donor fluorescent moieties that can be used with various acceptor fluorescent moieties in FRET technology include fluorescein, Lucifer Yellow, B-phycoerythrin, 9-acridineisothiocyanate, Lucifer Yellow VS, 4-acetamido-4′-isothiocyanatostilbene-2,2′-disulfonic acid, 7-diethylamino-3-(4′-isothiocyanatophenyl)-4-methylcoumarin, succinimdyl 1-pyrenebutyrate, and 4-acetamido-4′-isothiocyanatostilbene-2,2′-disulfonic acid derivatives. Representative acceptor fluorescent moieties, depending upon the donor fluorescent moiety used, include LC™-Red 640, LC™-Red 705, Cy5, Cy5.5, Lissamine rhodamine B sulfonyl chloride, tetramethyl rhodamine isothiocyanate, rhodamine x isothiocyanate, erythrosine isothiocyanate, fluorescein, diethylenetriamine pentaacetate, and other chelates of Lanthanide ions (e.g., Europium, or Terbium). Donor and acceptor fluorescent moieties can be obtained from, for example, Molecular Probes, Inc. (Eugene, Oreg.) or Sigma Chemical Co. (St. Louis, Mo.).

Donor and acceptor fluorescent moieties can be attached to probe oligonucleotides via linker arms. The length of each linker arm is important, as the linker arms will affect the distance between the donor and acceptor fluorescent moieties. The length of a linker arm for the purpose of the present invention is the distance in Angstroms (Å) from the nucleotide base to the fluorescent moiety. In general, a linker arm is from about 10 to about 25 Å in length. The linker arm may be of the kind described in WO 84/03285, for example. WO 84/03285 also discloses methods for attaching linker arms to a particular nucleotide base, as well as methods for attaching fluorescent moieties to a linker arm.

The amount of FRET corresponds to the amount of amplification product, which in turn corresponds to the amount of template nucleic acid present in the sample. Similarly, the amount of template nucleic acid corresponds to the amount of microbial organism present in the sample. Therefore, the amount of FRET produced when amplifying nucleic acid obtained from a biological sample can be correlated to the amount of a microorganism. Typically, the amount of a microorganism in a sample can be quantified by comparing to the amount of FRET produced from amplified nucleic acid obtained from known amounts of the microorganism (e.g., a standard curve). Accurate quantitation requires measuring the amount of FRET while amplification is increasing linearly. In addition, there must be an excess of probe in the reaction. Furthermore, the amount of FRET produced in the known samples used for comparison purposes can be standardized for particular reaction conditions, such that it is not necessary to isolate and amplify samples from every microorganism for comparison purposes.

As an alternative to FRET, an amplification product can be detected using, for example, a fluorescent DNA binding dye (e.g., SYBRGreenI® or SYBRGold® (Molecular Probes)). Upon interaction with an amplification product, such DNA binding dyes emit a fluorescent signal after excitation with light at a suitable wavelength. A double-stranded DNA binding dye such as a nucleic acid intercalating dye also can be used. When double-stranded DNA binding dyes are used, a melting curve analysis usually is performed for confirmation of the presence of the amplification product.

Melting curve analysis is an additional step that can be included in a cycling profile. Melting curve analysis is based on the fact that a nucleic acid sequence melts at a characteristic temperature (Tm), which is defined as the temperature at which half of the DNA duplexes have separated into single strands. The melting temperature of a DNA molecule depends primarily upon its nucleotide composition. A DNA molecule rich in G and C nucleotides has a higher Tm than one having an abundance of A and T nucleotides. The temperature at which the FRET signal is lost correlates with the melting temperature of a probe from an amplification product. Similarly, the temperature at which signal is generated correlates with the annealing temperature of a probe with an amplification product. The melting temperature(s) of probes from an amplification product can confirm the presence or absence of containing such sequences in a sample, and can be used to quantify the amount of a particular species. For example, a universal probe that hybridizes to a variable region within a sequence will have a Tm that depends upon the sequence to which it hybridizes. By observing a temperature-dependent, step-wise decrease in fluorescence of a sample as it is heated, the particular species in the sample can be identified and the relative amounts of the species in the sample can be determined.

Within each thermocycler run, control samples can be cycled as well. Positive control samples can amplify a nucleic acid control template (e.g., a nucleic acid other than the target nucleic acid) using, for example, control primers and control probes. Positive control samples also can amplify, for example, a plasmid construct containing a target nucleic acid molecule. Such a plasmid control can be amplified internally (e.g., within the sample) or in a separate sample run side-by-side with the test samples. Each thermocycler run also should include a negative control that, for example, lacks template DNA. Such controls are indicators of the success or failure of the amplification, hybridization and/or FRET reaction. Therefore, control reactions can readily determine, for example, the ability of primers to anneal with sequence-specificity and to initiate elongation, as well as the ability of probes to hybridize with sequence-specificity and for FRET to occur.

In one embodiment, methods of the invention include steps to avoid contamination. For example, an enzymatic method utilizing uracil-DNA glycosylase is described in U.S. Pat. Nos. 5,035,996, 5,683,896 and 5,945,313, and can be used to reduce or eliminate contamination between one thermocycler run and the next. In addition, standard laboratory containment practices and procedures are desirable when performing methods of the invention. Containment practices and procedures include, but are not limited to, separate work areas for different steps of a method, containment hoods, barrier filter pipette tips and dedicated air displacement pipettes. Consistent containment practices and procedures by personnel are necessary for accuracy in a diagnostic laboratory handling clinical samples.

It is understood that the present invention is not limited by the configuration of one or more commercially available instruments.

Fluorescent In Situ Hybridization (FISH)

In situ hybridization methods such as FISH also can be used to determine a microbial profile. In general, in situ hybridization methods provided herein include the steps of fixing a biological sample, hybridizing a probe to target DNA contained within the fixed biological sample, washing to remove non-specific binding, detecting the hybridized probe, and quantifying the amount of hybridized probe.

Typically, cells are harvested from a biological sample using standard techniques. For example, cells can be harvested by centrifuging a biological sample and resuspending the pelleted cells in, for example, phosphate-buffered saline (PBS). After re-centrifuging the cell suspension to obtain a cell pellet, the cells can be fixed in a solution such as an acid alcohol solution, an acid acetone solution, or an aldehyde such as formaldehyde, paraformaldehyde, or glutaraldehyde. For example, a fixative containing methanol and glacial acetic acid in a 3:1 ratio, respectively, can be used as a fixative. A neutral buffered formalin solution also can be used (e.g., a solution containing approximately 1% to 10% of 37-40% formaldehyde in an aqueous solution of sodium phosphate). Slides containing the cells can be prepared by removing a majority of the fixative, leaving the concentrated cells suspended in only a portion of the solution.

The cell suspension is applied to slides such that the cells do not overlap on the slide. Cell density can be measured by a light or phase contrast microscope. For example, cells harvested from a 20 to 100 ml urine sample typically are resuspended in a final volume of about 100 to 200 μl of fixative. Three volumes of this suspension (e.g., 3, 10, and 30 μl), are then dropped into 6 mm wells of a slide. The cellularity (i.e., the density of cells) in these wells is then assessed with a phase contrast microscope. If the well containing the greatest volume of cell suspension does not have enough cells, the cell suspension can be concentrated and placed in another well.

Probes for FISH are chosen for maximal sensitivity and specificity. Using a set of probes (e.g., two or more) can provide greater sensitivity and specificity than the use of any one probe. Probes typically are about 50 to about 2×10³ nucleotides in length (e.g., 50, 75, 100, 200, 300, 400, 500, 750, 1000, 1500, or 2000 nucleotides in length). Longer probes can comprise smaller fragments of about 100 to about 500 nucleotides in length. Probes that hybridize with locus-specific DNA can be obtained commercially from, for example, Vysis, Inc. (Downers Grove, Ill.), Molecular Probes, Inc. (Eugene, Oreg.), or from Cytocell (Oxfordshire, UK). Alternatively, probes can be made non-commercially from chromosomal or genomic DNA through standard techniques. For example, sources of DNA that can be used include genomic DNA, cloned DNA sequences, somatic cell hybrids that contain one, or a part of one, human chromosome along with the normal chromosome complement of the host, and chromosomes purified by flow cytometry or microdissection. The region of interest can be isolated through cloning, or by site-specific amplification via PCR. See, for example, Nath and Johnson, Biotechnic Histochem., 1998, 73(1):6-22, Wheeless et al., Cytometry, 1994, 17:319-326, and U.S. Pat. No. 5,491,224.

Probes for FISH typically are directly labeled with a fluorescent moiety (also referred to as a fluorophore), an organic molecule that fluoresces after absorbing light of lower wavelength/higher energy. The fluorescent moiety allows the probe to be visualized without a secondary detection molecule. After covalently attaching a fluorophore to a nucleotide, the nucleotide can be directly incorporated into a probe using standard techniques such as nick translation, random priming, and PCR labeling. Alternatively, deoxycytidine nucleotides within a probe can be transaminated with a linker. A fluorophore then can be covalently attached to the transaminated deoxycytidine nucleotides. See, U.S. Pat. No. 5,491,224. The amount of fluorophore incorporated into a probe can be known or determined, and this value in turn can be used to determine the amount of nucleic acid to which the probe binds. In conjunction with analysis of samples (e.g., a serial dilution of a sample) containing known numbers of microbial organisms, the number of microbial organisms in a biological or non-biological sample can be determined.

When more than one probe is used, fluorescent moieties of different colors can be chosen such that each probe in the set can be distinctly visualized and quantitated. For example, a combination of the following fluorophores may be used: 7-amino-4-methylcoumarin-3-acetic acid (AMCA), Texas Red™ (Molecular Probes, Inc.), 5-(and-6)-carboxy-X-rhodamine, lissamine rhodamine B, 5-(and-6)-carboxyfluorescein, fluorescein-5-isothiocyanate (FITC), 7-diethylaminocoumarin-3-carboxylic acid, tetramethylrhodamine-5-(and-6)-isothiocyanate, 5-(and-6)-carboxytetramethylrhodamine, 7-hydroxycoumarin-3-carboxylic acid, 6-[fluorescein 5-(and-6)-carboxamido]hexanoic acid, N-(4,4-difluoro-5,7-dimethyl-4-bora-3a,4a diaza-3-indacenepropionic acid, eosin-5-isothiocyanate, erythrosin-5-isothiocyanate, and Cascade™ blue acetylazide (Molecular Probes, Inc.). Probes can be viewed with a fluorescence microscope and an appropriate filter for each fluorophore, or by using dual or triple band-pass filter sets to observe multiple fluorophores. See, for example, U.S. Pat. No. 5,776,688. Alternatively, techniques such as flow cytometry can be used to examine and quantitate the hybridization pattern of the probes.

Probes also can be indirectly labeled with biotin or digoxygenin, or labeled with radioactive isotopes such as ³²P and ³H, although secondary detection molecules or further processing then may be required to visualize the probes and quantify the amount of hybridization. For example, a probe indirectly labeled with biotin can be detected and quantitated using avidin conjugated to a detectable enzymatic marker such as alkaline phosphatase or horseradish peroxidase. Enzymatic markers can be detected and quantitated in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase.

Prior to in situ hybridization, the probes and the chromosomal DNA contained within the cell each are denatured. Denaturation typically is performed by incubating in the presence of high pH, heat (e.g., temperatures from about 70° C. to about 95° C.), organic solvents such as formamide and tetraalkylammonium halides, or combinations thereof. For example, chromosomal DNA can be denatured by a combination of temperatures above 70° C. (e.g., about 73° C.) and a denaturation buffer containing 70% formamide and 2×SSC (0.3 M sodium chloride and 0.03 M sodium citrate). Denaturation conditions typically are established such that cell morphology is preserved. Probes can be denatured by heat (e.g., by heating to about 73° C. for about five minutes).

After removal of denaturing chemicals or conditions, probes are annealed to the chromosomal DNA under hybridizing conditions. “Hybridizing conditions” are conditions that facilitate annealing between a probe and target chromosomal DNA. Hybridization conditions vary, depending on the concentrations, base compositions, complexities, and lengths of the probes, as well as salt concentrations, temperatures, and length of incubation. The higher the concentration of probe, the higher the probability of forming a hybrid. For example, in situ hybridizations typically are performed in hybridization buffer containing 1-2×SSC, 50% formamide, and blocking DNA to suppress non-specific hybridization. In general, hybridization conditions, as described above, include temperatures of about 25° C. to about 55° C., and incubation times of about 0.5 hours to about 96 hours. More particularly, hybridization can be performed at about 32° C. to about 40° C. for about 2 to about 16 hours.

Non-specific binding of probes to DNA outside of the target region can be removed by a series of washes. The temperature and concentration of salt in each wash depend on the desired stringency. For example, for high stringency conditions, washes can be carried out at about 65° C. to about 80° C., using 0.2× to about 2×SSC, and about 0.1% to about 1% of a non-ionic detergent such as Nonidet P-40 (NP40). Stringency can be lowered by decreasing the temperature of the washes or by increasing the concentration of salt in the washes.

mRNA-Based Assays

Alternatively, in order to test for the presence or absence of, or measure the level of, a specific mRNA in a sample, e.g., a sample comprising cells, the cells can be lysed and total RNA can be purified or semi-purified from lysates by any of a variety of methods known in the art. Methods of detecting or measuring levels of particular mRNA transcripts are also familiar to those in the art. Such assays include, without limitation, hybridization assays using detectably labeled specific nucleic acid (DNA or RNA) probes and quantitative or semi-quantitative RT-PCR methodologies employing appropriate oligonucleotide primers. Additional methods for quantitating mRNA in cell lysates include RNA protection assays and serial analysis of gene expression (SAGE). Alternatively, qualitative, quantitative, or semi-quantitative in situ hybridization assays can be carried out using, for example, samples such as tissue sections or unlysed cell suspensions, and detectably (e.g., fluorescently, isotopically, or enzymatically) labeled DNA or RNA probes.

Representative Polypeptide-Based Assays

The invention also features polypeptide-based assays. A microbial polypeptide can be used as a universal target to determine the presence or absence of one or more microbes, and further used as species-specific targets and/or probes for the identification and classification of specific microbes. Such assays can be used on their own or in conjunction with other procedures (e.g., nucleic acid-based assays) to detect and monitor animal by-products.

In the assays of the invention, the presence or absence of a microbial polypeptide is detected and/or its level is measured.

Methods of detecting or measuring the levels of a protein of interest (e.g., a cpn60 protein, or cpn60-specific polypeptides) in cells are known in the art. Many such methods employ antibodies (e.g., polyclonal antibodies or mAbs) that bind specifically to the protein.

Antibodies and Antibody-Based Assays

Antibodies having specific binding affinities for a microbial polypeptide may be produced through standard methods. As used herein, the terms “antibody” or “antibodies” include intact molecules as well as fragments thereof which are capable of binding to an epitopic determinant of a specific polypeptide. The term “epitope” refers to an antigenic determinant on an antigen to which the paratope of an antibody binds. Epitopic determinants usually consist of chemically active surface groupings of molecules such as amino acids or sugar side chains, and typically have specific three-dimensional structural characteristics, as well as specific charge characteristics. Epitopes generally have at least five contiguous amino acids (a continuous epitope), or alternatively can be a set of noncontiguous amino acids that define a particular structure (e.g., a conformational epitope). The terms “antibody” and “antibodies” include polyclonal antibodies, monoclonal antibodies, humanized or chimeric antibodies, single chain Fv antibody fragments, Fab fragments, and F(ab)₂ fragments.

Antibodies may be specific for a particular polypeptide specific to a genus or species of microbe. Alternatively, they may be cross-reactive with polypeptides common to more than one genera or species. For example, such antibodies may bind to common epitopes present in homologs from different microbes. As used herein, such antibodies with specificity for a polypeptide from more than one microbe are termed “universal” antibodies. For example, certain antibodies may bind to common epitopes present in homologs from a number of different microbes. Certain of such antibodies thus may be termed able to detect the presence or absence of any of a large number of microbes in a sample.

In certain embodiments of the method described herein, depending on the animal by-product and the purpose for monitoring, it may be sufficient to determine simply whether or not any microbe is present, and optionally the relative concentration or amount of the microbe. Such detection may occur through, e.g., the use of one or more “universal” antibodies.

In other embodiments, the identification of the particular microbe may be preferred. Accordingly, an antibody having specific binding affinity for a polypeptide from a particular organism may be employed, either alone or in conjunction with a universal antibody; such antibodies are referred to as “specific” antibodies herein. The universal and specific antibodies may be employed simultaneously or in series. For example, a universal antibody may be used as a first screen to determine the presence or absence of a polypeptide. Subsequently, a specific antibody, such as one specific for a polypeptide from a particular microbe, e.g., Campylobacter jejuni, may be employed. In such assays, monoclonal antibodies may be particularly useful (e.g., sensitive) to identify polypeptides of a particular microbe.

In general, a protein of interest (e.g., a protein against which one wishes to prepare antibodies) is produced recombinantly, by chemical synthesis, or by purification of the native protein, and then used to immunize animals. As used herein, an intact protein or a polypeptide fragment thereof may be employed, provided that the polypeptide is capable of generating the desired immune response. See, for example, WO 200265129 for examples of epitopic sequences that bind to human antibodies against Chlamydia trachomatis; such epitopic sequences may be useful in generating antibodies against Chlamydia spp. for use in the present invention. See also U.S. Pat. No. 6,497,880, which sets forth nucleic acid sequences, amino acid sequences, expression vectors, purified proteins, antibodies, etc. specific to Aspergillus fumigatus and Candida glabrata. Purified Aspergillus fumigatus and Candida glabrata cpn60 proteins, for example, or proteolytically or synthetically generated fragments thereof, can be used to immunize animals to generate antibodies for use in the methods of the present invention. Finally, see WO 02/57784, disclosing substantially purified Chlamydia hsp60 (cpn60) polypeptides. Such polypeptides may also be used to generate antibodies for use in the methods of the present invention.

As discussed previously, one may wish to prepare universal or specific antibodies to a protein or polypeptide. A polypeptide may be used to generate a universal antibody, for example, if it maintains an epitope that is common to at least two proteins, or, e.g., to all proteins from each species that one wishes to detect. Alternatively, a protein or polypeptide may be used to generate antibodies specific for a particular protein or polypeptide present in a specific microbe, e.g., only Campylobacter jejuni.

Various host animals including, for example, rabbits, chickens, mice, guinea pigs, and rats, can be immunized by injection of the protein of interest. Adjuvants can be used to increase the immunological response depending on the host species and include Freund's adjuvant (complete and incomplete), mineral gels such as aluminum hydroxide, surface-active substances such as lysolecithin, pluronic polyols, polyanions, peptides, oil emulsions, keyhole limpet hemocyanin (KLH), and dinitrophenol. Polyclonal antibodies are heterogenous populations of antibody molecules that are specific for a particular antigen, which are contained in the sera of the immunized animals. Monoclonal antibodies, which are homogeneous populations of antibodies to a particular epitope contained within an antigen, can be prepared using standard hybridoma technology. In particular, monoclonal antibodies can be obtained by any technique that provides for the production of antibody molecules by continuous cell lines in culture such as described by Kohler, G. et al., Nature, 1975, 256:495, the human B-cell hybridoma technique (Kosbor et al., Immunology Today, 1983, 4:72; Cole et al., Proc. Natl. Acad. Sci. USA, 1983, 80:2026), and the EBV-hybridoma technique (Cole et al., “Monoclonal Antibodies and Cancer Therapy”, Alan R. Liss, Inc., 1983, pp. 77-96). Such antibodies can be of any immunoglobulin class including IgG, IgM, IgE, IgA, IgD, and any subclass thereof. The hybridoma producing the monoclonal antibodies of the invention can be cultivated in vitro or in vivo.

A chimeric antibody is a molecule in which different portions are derived from different animal species, such as those having a variable region derived from a murine monoclonal antibody and a human immunoglobulin constant region. Chimeric antibodies can be produced through standard techniques.

Antibody fragments that have specific binding affinity for a polypeptide can be generated by known techniques. For example, such fragments include, but are not limited to, F(ab′)₂ fragments that can be produced by pepsin digestion of the antibody molecule, and Fab fragments that can be generated by reducing the disulfide bridges of F(ab′)₂ fragments. Alternatively, Fab expression libraries can be constructed. See, for example, Huse et al., 1989, Science, 246:1275. Single chain Fv antibody fragments are formed by linking the heavy and light chain fragments of the Fv region via an amino acid bridge (e.g., 15 to 18 amino acids), resulting in a single chain polypeptide. Single chain Fv antibody fragments can be produced through standard techniques. See, for example, U.S. Pat. No. 4,946,778.

Once produced, antibodies or fragments thereof are tested for recognition of the protein or polypeptide by standard immunoassay methods including, for example, ELISA techniques, countercurrent immuno-electrophoresis (CIEP), radioimmunassays (RIA), radioimmunoprecipitations, dot blots, inhibition or competition assays, sandwich assays, immunostick (dipstick) assays, immunochromatographic assays, immunofiltration assays, latex beat agglutination assays, immunofluoroescent assays, biosensor assays. See, Short Protocols in Molecular Biology, Chapter 11, Green Publishing Associates and John Wiley & Sons, Edited by Ausubel, F. M et al., 1992; Antibodies: A Laboratory Manual, Harlow and Lane (eds.), Cold Spring Harbor Laboratory Press, 1988; and U.S. Pat. Nos. 4,376,110; 4,486,530; and 6,497,880. Antibodies or fragments can also be tested for their ability to react universally, e.g., with proteins or polypeptides from more than one genus or species of microbe or specifically with a particular protein from a specific organism.

In antibody assays, the antibody itself or a secondary antibody that binds to it can be detectably labeled. Alternatively, the antibody can be conjugated with biotin, and detectably labeled avidin (a protein that binds to biotin) can be used to detect the presence of the biotinylated antibody. Combinations of these approaches (including “multi-layer” assays) familiar to those in the art can be used to enhance the sensitivity of assays. Some of these assays (e.g., immunohistological methods or fluorescence flow cytometry) can be applied to histological sections or unlysed cell suspensions. The methods described below for detecting a polypeptide in a liquid sample can also be used to detect a polypeptide in cell lysates.

Methods of detecting a polypeptide in a liquid sample generally involve contacting a sample of interest with an antibody that binds to the polypeptide and testing for binding of the antibody to a component of the sample. In such assays the antibody need not be detectably labeled and can be used without a second antibody. For example, an antibody having binding affinity for a polypeptide may be bound to an appropriate solid substrate and then exposed to the sample. Binding of a polypeptide to the antibody on the solid substrate may be detected by exploiting the phenomenon of surface plasmon resonance, which results in a change in the intensity of surface plasmon resonance upon binding that can be detected qualitatively or quantitatively by an appropriate instrument, e.g., a Biacore apparatus (Biacore International AB, Rapsgatan, Sweden).

Moreover, assays for detection of a polypeptide in a liquid sample can involve the use, for example, of: (a) a single antibody specific for a polypeptide that is detectably labeled; (b) an unlabeled antibody that is specific for a polypeptide and a detectably labeled secondary antibody; or (c) a biotinylated antibody specific for a polypeptide and detectably labeled avidin. In addition, combinations of these approaches (including “multi-layer” assays) familiar to those in the art can be used to enhance the sensitivity of assays. In these assays, the sample or an aliquot of the sample suspected of containing a microbe can be immobilized on a solid substrate, such as a nylon or nitrocellulose membrane, by, for example, “spotting” an aliquot of the liquid sample or by blotting of an electrophoretic gel on which the sample or an aliquot of the sample has been subjected to electrophoretic separation. The presence or amount of polypeptide on the solid substrate is then assayed using any of the above-described forms of the polypeptide specific antibody and, where required, appropriate detectably labeled secondary antibodies or avidin.

The invention also features “sandwich” assays. In these sandwich assays, instead of immobilizing samples on solid substrates by the methods described above, any polypeptide that may be present in a sample can be immobilized on the solid substrate by, prior to exposing the solid substrate to the sample, conjugating a second (“capture”) antibody (polyclonal or mAb) specific for the polypeptide to the solid substrate by any of a variety of methods known in the art. In exposing the sample to the solid substrate with the second antibody specific for the polypeptide bound to it, any polypeptide in the sample (or sample aliquot) will bind to the second antibody on the solid substrate. The presence or amount of polypeptide bound to the conjugated second antibody is then assayed using a “detection” antibody specific for a polypeptide by methods essentially the same as those described above using a single antibody specific for a polypeptide. It is understood that in these sandwich assays, the capture antibody should not bind to the same epitope (or range of epitopes in the case of a polyclonal antibody) as the detection antibody. Thus, if a mAb is used as a capture antibody, the detection antibody can be either: (a) another mAb that binds to an epitope that is either completely physically separated from or only partially overlaps with the epitope to which the capture mAb binds; or (b) a polyclonal antibody that binds to epitopes other than or in addition to that to which the capture mAb binds. On the other hand, if a polyclonal antibody is used as a capture antibody, the detection antibody can be either (a) a mAb that binds to an epitope to that is either completely physically separated from or partially overlaps with any of the epitopes to which the capture polyclonal antibody binds; or (b) a polyclonal antibody that binds to epitopes other than or in addition to that to which the capture polyclonal antibody binds. Assays that involve the used of a capture and detection antibody include sandwich ELISA assays, sandwich Western blotting assays, and sandwich immunomagnetic detection assays.

Suitable solid substrates to which the capture antibody can be bound include, without limitation, the plastic bottoms and sides of wells of microtiter plates, membranes such as nylon or nitrocellulose membranes, and polymeric (e.g., without limitation, agarose, cellulose, or polyacrylamide) beads or particles. It is noted that antibodies bound to such beads or particles can also be used for immunoaffinity purification of polypeptides. Dipstick/immunostick formats can employ a solid phase, e.g., polystyrene, paddle or dispstick.

Methods of detecting or for quantifying a detectable label depend on the nature of the label and are known in the art. Appropriate labels include, without limitation, radionuclides (e.g. ¹²⁵I, ¹³¹I, ³⁵S, ³H, ³²P, ³³P, or ¹⁴C), fluorescent moieties (e.g., fluorescein, rhodamine, or phycoerythrin), luminescent moieties (e.g., Qdot™ nanoparticles supplied by the Quantum Dot Corporation, Palo Alto, Calif.), compounds that absorb light of a defined wavelength, or enzymes (e.g., alkaline phosphatase or horseradish peroxidase). The products of reactions catalyzed by appropriate enzymes can be, without limitation, fluorescent, luminescent, or radioactive, or they may absorb visible or ultraviolet light. Examples of detectors include, without limitation, x-ray film, radioactivity counters, scintillation counters, spectrophotometers, colorimeters, fluorometers, luminometers, and densitometers.

The methods of the present invention may employ a control sample. In assays to detect the presence or absence of a microbe, the concentration of a microbial polypeptide may be compared to a control sample. The control sample may be taken from the same high-risk environment, e.g., in a different location known to be uncontaminated, or can be a control sample taken from a non-high-risk environment. Alternatively, the control sample may be taken from the same location of a high-risk environment but at an earlier or later time-point when the location was known to be uncontaminated. A significantly higher concentration of polypeptide in the suspect sample relative to the control sample would indicate the presence of a microbe.

It is understood that, while the above descriptions of the diagnostic assays may refer to assays on particular samples, the assays can also be carried out on any of the other fluid or solubilized samples listed herein, such as water samples or buffer samples (e.g., buffer used to extract a sample from a swab).

Other Polypeptide-Based Detection Assays

The present invention also contemplates the use of other analytical techniques for detecting microbial polypeptides. Recent analytical instrumentation and methodology advances that have arisen in the context of proteomics research are applicable in the methods of the present invention. See, generally, PR Jungblut, “Proteome Analysis of Bacterial Pathogens,” Microbes & Infection 3 (2001): 831-840; G MacBeath and SL Schreiber, “Printing Proteins as Microarrays for High-Throughput Function Determination,” Science 289 (2000): 1760-1763; J Madoz-Gdrpide, H Wang, and D E Misek, “Protein-Based Microarrays: A Tool for Probing the Proteome of Cancer Cells and Tissues,” Proteomics 1 (2001): 1279-1287; S Patterson, “Mass Spectrometry and Proteomics,” Physiological Genomics 2 (2000): 59-65; and A Schevchenko et al., “Maldi Quadrupole Time-of-Flight Mass Spectrometry: A Powerful Tool for Proteomic Research,” Analytical Chemistry 72 (2000):2132-2141.

Mass-spectrophotometric techniques have been increasingly used to detect and identify proteins and protein fragments at low levels, e.g., fmol or pmol. Mass spectrometry has become a major analytical tool for protein and proteomics research because of advancements in the instrumentation used for biomolecular ionization, electrospray ionization (ESI), and matrix-assisted laser desorption-ionization (MALDI). MALDI is usually combined with a time-of-flight (TOF) mass analyzer. Typically, 0.5 μl of sample that contains 1-10 pmol of protein or peptide is mixed with an equal volume of a saturated matrix solution and allowed to dry, resulting in the co-crystallization of the analyte with the matrix. Matrix compounds that are used include sinapic acid and α-hydroxycinnamic acid. The co-crystallized material on the target plate is irradiated with a nitrogen laser pulse, e.g., at a wavelength of 337 nm, to volatilize and ionize the protein or peptide molecules. A strong acceleration field is switched on, and the ionized molecules move down the flight tube to a detector. The amount of time required to reach the detector is related to the mass-to-charge ratio. Proteolytic mass mapping and tandem mass spectrometry, when combined with searches of protein and protein fragment databases, can also be employed to detect and identify polypeptides. See, for example, Downard, “Contributions of mass spectrometry to structural immunology,” J. Mass. Spectrom. 35:493-503 (2000).

Biomolecular interaction analysis mass spectrometry (BIA-MS) is another suitable technique for detecting interactions between polypeptides and antibodies. This technology detects molecules bound to a ligand that is covalently attached to a surface. As the density of biomaterial on the surface increases, changes occur in the refractive index at the solution or surface interface. This change in the refractive index is detected by varying the angle or wavelength at which the incident light is absorbed at the surface. The difference in the angle or wavelength is proportional to the amount of material bound on the surface, giving rise to a signal that is termed surface plasmon resonance (SPR), as discussed previously. See, for example, R W Nelson et al., “BIA/MS of Epitope-Tagged Peptides Directly from E. coli Lysate: Multiplex Detection and Protein Identification at Low-Femtomole to Subfemtomole Levels,” Analytical Chemistry 71 (1999): 2858-2865; see also D Nedelkov and R W Nelson, “Analysis of Native Proteins from Biological Fluids by Biomolecular Interaction Analysis Mass Spectrometry (BIA/MS): Exploring the Limit of Detection, Identification of Non-Specific Binding and Detection of Multiprotein Complexes,” Biosensors and Bioelectronics 16 (2001): 1071-1078.

The SPR biosensing technology has been combined with MALDI-TOF mass spectrometry for the desorption and identification of biomolecules. In a chip-based approach to BIA-MS, a ligand, e.g., an antibody, is covalently immobilized on the surface of a chip. A tryptic digest of solubilized proteins from a sample is routed over the chip, and the relevant peptides or polypeptides, are bound by the ligand. After a washing step, the eluted peptides are analyzed by MALDI-TOF mass spectrometry. The system may be a fully automated process and is applicable to detecting and characterizing proteins present in complex biological fluids and cell extracts at low- to subfemtomol levels.

Mass spectrometers useful for such applications are available from Applied Biosystems (Foster City, Calif.); Bruker Daltronics (Billerica, Mass.) and Amersham Pharmacia (Sunnyvale, Calif.).

Other suitable techniques for use in the present invention include “Multidimensional Protein Identification Technologies.” Cells are fractionally solubilized and digested, e.g., sequentially with endoproteinase Lys-C and immobilized trypsin. The samples are then subjected to multidimensional protein identification technology (MUDPIT), which involves a sequential separation of the peptide fragments by on-line biphasic microcapillary chromatography (e.g., strong ion exchange, then C-18 separation), followed by tandem mass spectrometry (MS-MS). See, for example, Washburn et al., “Large-Scale Analysis of the Yeast Proteome by Multidimensional Protein Identification Technology,” Nature Biotechnology 19:242-247 (2001).

Articles of Manufacture

The invention also provides articles of manufacture. Articles of manufacture can include at least one oligonucleotide, as well as instructions for collecting a sample from an animal by-product and using the oligonucleotide(s) to determine the presence, absence, or amount of microbial contamination in a sample from an animal by-product. An embodiment useful in an article of manufacture of the invention can be an oligonucleotide that is complementary to nucleic acid sequences from one or a few number of microbial species, or can be an oligonucleotide that is complementary to nucleic acid sequences from more than one microbial species or across genera.

In one embodiment, the oligonucleotide(s) are attached to a microarray (e.g., a GeneChip®, Affymetrix, Santa Clara, Calif.). In another embodiment, an article of manufacture can include one or more oligonucleotide primers and one or more oligonucleotide probes. Such primers and probes can be used, for example, in real-time amplification reactions to amplify and simultaneously detect amplification products.

Suitable oligonucleotides include those that are complementary to highly conserved regions of a nucleic acid sequence and that flank a variable region. Such universal primers can be used to specifically amplify these variable regions, thereby providing a sequence with which to identify microorganisms. Examples of oligonucleotide primers, which are universal primers to microbial cpn60 sequences, include the following:

5′-GAIIIIGCIGGIGA(T/C)GGIACIACIAC-3′ (SEQ ID NO:1); and

5′-(T/C)(T/G)I(T/C)(T/G)ITCICC(A/G)AAICCIGGIGC(T/C)TT-3′ (SEQ ID NO:2).

Suitable oligonucleotides also include those that are complementary to species-specific sequences, and thus result in an amplification product only if a particular species is present in the sample.

Similar to oligonucleotide primers, oligonucleotide probes generally are complementary to the target sequences. Oligonucleotide probes can be designed to hybridize universally to target sequences, or can be designed for species-specific hybridization to a variable region of the target sequences.

An article of manufacture of the invention can further include additional components for carrying out amplification reactions and/or reactions, for example, on a microarray. Articles of manufacture for use with PCR reactions can include nucleotide triphosphates, an appropriate buffer, and a polymerase. An article of manufacture of the invention also can include appropriate reagents for detecting amplification products. For example, an article of manufacture can include one or more restriction enzymes for distinguishing amplification products from different species of microorganism, or can include fluorophore-labeled oligonucleotide probes for real-time detection of amplification products.

It will be appreciated by those of ordinary skill in the art that different articles of manufacture can be provided to evaluate microbes from different types of animal by-products. For example, fishmeal may contain a different community of microbes than that of poultry meal. Therefore, an article of manufacture designed to evaluate the microbes in fishmeal may have a different set of controls or a different set of species-specific hybridization probes than that designed for use with poultry meal. Alternatively, a more generalized article of manufacture can be used to evaluate the microbes in a number of different animal by-products.

In addition, articles of manufacture are provided that include at least one microbial antibody, as well as instructions for collecting a sample from an animal by-product and using the antibody to detect the presence, absence, or amount of microbial contamination in an animal by-product.

In one embodiment, one or more microbial antibodies are attached to a microarray (e.g., a 96-microwell plate). For example, a microarray format may include a variety of universal and specific capture antibodies; the universal and specific antibodies may each be located at a different well location. The article of manufacture may also include the appropriate detection antibodies, if necessary, and appropriate reagents for binding of a microbial polypeptide to one or more capture antibodies (e.g., enzymes, substrates, buffers, and controls).

In another embodiment, an article of manufacture can include one or more microbial antibodies attached to a dipstick. Such dipsticks can be used, for example, to detect microbial polypeptides in a liquid sample.

The invention will be further described in the following examples, which do not limit the scope of the invention described in the claims.

### EXAMPLES

### Example 1

### Testing Fishmeal for Microbial Contamination

20 samples of approximately 1 cm³ fishmeal are obtained from the quality control laboratory of a poultry production unit. The fishmeal is used as feed for the chickens, and is tested for viable microbial load. The sample is macerated and serially diluted in phosphate buffer (pH 7.0). The dilutions are plated onto plates containing plate count agar medium. The plates are grown at 37° C. overnight, and evaluated for microbial growth. The plates are left at 37° C. for an additional 24-48 hours and evaluated daily for microbial growth. The number of colonies from each dilution sample is counted with a plate reader after 48 hours of growth. In addition, the dilution samples are also plated onto brilliant green agar to detect any Salmonella spp. present in the sample. Brilliant green agar plates are incubated aerobically at 37° C. for 24 to 48 hours. Red colonies are presumptive Salmonella spp. If low numbers of colonies are observed, the undiluted sample is enriched in selenite cystine broth overnight, followed by plating on brilliant green agar. The dilution samples are also plated on bile esculin agar to detect the presence of Enterococcus spp. The bile exculin agar plates are incubated anaerobically at 37° C. for 24 to 48 hours. The dilution samples are additionally plated on MacConkey agar to detect the presence of E. coli and grown aerobically at 37° C. for 24 to 48 hours. Colony numbers on each plate are counted using a plate reader.

### Example 2

### Quantitating Microbial Organisms Using cpn60 Universal Primers and a cpn60 Universal Probe

A biological sample is obtained from poultry GIT and genomic DNA is extracted using standard methods (Diagnostic Molecular Microbiology: Principles and Applications (supra)). Real-time PCR is conducted using universal cpn60 primers having the nucleotide sequences set forth in SEQ ID NO:1 and SEQ ID NO:2, and a universal cpn60 probe having the sequence 5′-GACAAAGTCGGTAAAGAAGGCGTTATCA-3′ (SEQ ID NO:3), labeled at the 5′ end with fluorescein (fluorophore; Molecular Probes, Inc.) and at the 3′ end with dabcyl (quencher; (4-(4′-dimethylaminophenylazo)benzoic acid) succinimidyl ester; Molecular Probes, Inc.). This probe binds to a variable region of the cpn60 gene from numerous microbial species; thus the Tm of the probe from an amplification product varies depending upon the nucleotide sequence within the amplification product to which the probe hybridizes.

The PCR reaction contains 3 μL extracted DNA, 1 μM each universal cpn60 primer, 340 nM universal cpn60 probe, 2.5 units Amplitaq Gold DNA polymerase (Perkin Elmer), 0.25 mM each deoxyribonucleotide, 3.5 mM MgCl₂, 50 mM KCl, and 10 mM Tris-HCl, pH 8.0 in a total reaction volume of 50 μL. PCR conditions include an initial incubation at 95° C. for 10 minutes to activate the Amplitaq Gold DNA polymerase, followed by 40 cycles of 30 seconds at 95° C., 60 seconds at 50° C., and 30 seconds at 72° C. Fluorescence is monitored during the 50° C. annealing steps throughout the 40 cycles. After the cycling steps are complete, the melting temperature of the universal probe from the amplification products is analyzed. As the temperature is increased, the universal probe is released from the amplification product from each species' cpn60 sequence at a specific temperature, corresponding to the Tm of the universal probe and the cpn60 sequence of the particular species. The step-wise loss of fluorescence at particular temperatures is used to identify the particular species present, and the loss in fluorescence of each step compared to the total amount of fluorescence correlates with the relative amount of each microorganism.

### Example 3

### Dipstick ELISA Assay for Streptococcus

A polystyrene dipstick containing two horizontal bands is constructed: one band consists of broadly reactive, polyclonal capture antibodies against cpn60 proteins from Streptococcus spp., while the other band is an internal control consisting of horseradish peroxidase. The assay is performed by making serial dilutions (1:2, 1:5, 1:10, etc.) of a liquid sample taken from a high risk environment (e.g., a urine sample or a blood sample) directly into a detection reagent and incubating a wetted dipstick in these dilutions for 5 minutes, and then adding an indicator to detect binding of cpn60 proteins to the capture (and detection) antibodies. The detection reagent includes a suitable buffer and secondary cpn60 Streptococcus detection antibodies labeled with horseradish peroxidase. The indicator can be a chromogenic horseradish peroxidase substrate, such as 2,2′-AZINO-bis 3-ethylbenziazoline-6-sulfonic acid, or ABTS. ABTS is considered a safe, sensitive substrate for horseradish peroxidase that produces a blue-green color upon enzymatic activity that can be quantitated at 405-410 nm. At the end of the incubation and indicator steps, the dipstick is rinsed with water (e.g., deionized water) and examined for staining of the antibody band by visual inspection. Staining of the antibody band reveals the presence of Streptococcus spp. in the sample. The internal control band provides a check on the integrity of the detection reagent.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for determining the presence, absence, or amount of microbial contamination in an animal by-product, comprising:
providing a sample from said animal by-product; and
determining the presence, absence, or amount of microbial contamination in said sample,
wherein the presence of microbial contamination in said sample indicates the presence of microbial contamination in said animal by-product.

2. The method of claim 1, wherein microbes are taxonomically and phylogenetically identified.

3. The method of claim 2, wherein said microbes are selected from the group consisting of bacteria, fungi, viruses, and protozoa.

4. The method of claim 3, wherein said bacterial microbe is selected from the group consisting of the Staphylococcus, Streptococcus, Pseudomonas, Escherichia, Bacillus, Brucella, Chlamydia, Clostridium, Shigella, Mycobacterium, Agrobacterium, Bartonella, Borellia, Bradyrhizobium, Ehrlichia, Haemophilus, Helicobacter, Heliobacter, Lactobacillus, Neisseria, Rhizobium, Streptomyces, Synechococcus, Zymomonas, Synechocyotis, Mycoplasma, Yersinia, Vibrio, Burkholderia, Franciscella, Legionella, Salmonella, Bifidobacterium, Enterococcus, Enterobacter, Citrobacter, Bacteroides, Prevotella, Xanthomonas, Xylella, and Campylobacter genera.

5. The method of claim 3, wherein said fungal microbe is selected from the group consisting of the Aspergillus, Colletrotrichum, Cochliobolus, Helminthosporium, Microcyclus, Puccinia, Pyricularia, Deuterophoma, Monilia, Candida, and Saccharomyces genera.

6. The method of claim 3, wherein said viral microbe is from the Coronaviridae genus.

7. The method of claim 3, wherein said protozoan microbe is selected from the group consisting of the Acanthamoeba, Cryptosporidium, and Tetrahymena genera.

8. The method of claim 1, wherein said animal by-product is selected from the group consisting of hair, feathers, bone, viscera, blood, heads, feet, beaks, offal, and meat scraps.

9. The method of claim 1, wherein said animal by-product is a processed animal by-product.

10. The method of claim 9, wherein said processed animal by-product is selected from the group consisting of fishmeal, feathermeal, blood meal, poultry litter, and bone meal.

11. The method of claim 1, wherein said sample is selected from the group consisting of fishmeal, feathermeal, bone meal, blood meal, and offal.

12. The method of claim 1, wherein said sample is a plurality of samples.

13. The method of claim 12, wherein said plurality of samples are pooled.

14. The method of claim 1, wherein said determining comprises microbial culturing and colony identification.

15. The method of claim 1, wherein said determining comprises histological analysis.

16. The method of claim 1, wherein said determining comprises immunological analysis.

17. The method of claim 1, wherein said determining comprises genetic fingerprinting.

18. The method of claim 1, wherein said determining comprises ribosomal genotyping.

19. The method of claim 1, wherein said determining comprises cpn60 genotyping.

20. The method of claim 1, further comprising:
providing a control sample; and
determining the amount of microbes in said control sample.

21. The method of claim 20, wherein said control sample is a known amount of microbes.

22. A method for monitoring animal by-products before, during, or after processing said animal by-product, comprising:
providing a sample from said animal by-product;
determining the presence, absence, or amount of microbial contamination in said sample; and
tailoring said processing based upon said presence, absence, or amount of said microbial contamination.

23. The method of claim 22, wherein when said sample is obtained before said processing of said animal by-product, said tailoring comprises:
abandoning said processing; or
expediting said processing.

24. The method of claim 22, wherein when said sample is obtained during said processing of said animal by-product, said tailoring comprises:
abandoning said processing;
expediting said processing; or
repeating all or a portion of said processing.

25. The method of claim 22, wherein when said sample is obtained after said processing of said animal by-product, said tailoring comprises:
repeating all or a portion of said processing.

26. The method of claim 22, wherein said tailoring is based on the presence or absence of said microbial contamination.

27. The method of claim 22, wherein said tailoring is based on the amount of said microbial contamination.

28. The method of claim 22, wherein said determining is specific for one or a few species or genera of microbes.

29. The method of claim 22, wherein said microbial contamination is by one or a few species or genera of microbes.

30. The method of claim 22, wherein said determining is universal for many species or genera of microbes.

31. The method of claim 22, wherein said microbial contamination is by many species or genera of microbes.

32. An article of manufacture, comprising:
at least one microbial antibody, wherein said antibody is attached to a solid support; and
instructions for collecting a sample from an animal by-product and determining the presence, absence, or amount of microbial contamination in said sample.

33. The article of manufacture of claim 32, further comprising an indicator molecule.

34. The article of manufacture of claim 32, wherein said solid support is a dipstick.

35. An article of manufacture, comprising:
at least one oligonucleotide, wherein said oligonucleotide is complementary to nucleic acid sequences from one or a few number of microbial species; and
instructions for collecting a sample from an animal by-product and determining the presence, absence, or amount of microbial contamination in said sample.

36. An article of manufacture, comprising:
at least one oligonucleotide, wherein said oligonucleotide is complementary to nucleic acid sequences from microbial species of at least two genera; and
instructions for collecting a sample from an animal by-product and determining the presence, absence, or amount of microbial contamination in said sample.
